(19) United States
(12) Patent Application Publication
(10) Pub. No.: US 2024/0382092 A1
(43) Pub. Date: Nov. 21, 2024

(54) DEVICE AND METHOD FOR DETECTION

(71) Applicant: Wistron Corp., New Taipei City (TW)

(72) Inventor: Yao Tsung CHANG, New Taipei City (TW)

(21) Appl. No.: 18/337,155

(22) Filed: Jun. 19, 2023

(30) Foreign Application Priority Data
May 17, 2023 (TW) 112118258

(51) Int. Cl. A61B 5/00; A61B 5/05

(52) U.S. Cl. CPC A61B 5/002; A61B 5/0008; A61B 5/05; A61B 2503/04; G08C 17/02

(57) ABSTRACT

A detection device for detecting an object with a sensor includes a transmitter module, a receiver module, a processing module, and a computing module. The transmitter module transmits an RF (Radio Frequency) signal to the object. The receiver module receives a reflective signal from the object. The reflective signal includes a feature signal and a sense signal. The processing module converts the reflective signal into an integrated digital signal. The computing module determines the integrated digital signal into first digital information and second digital information. The first digital information corresponds to the feature signal. The second digital information corresponds to the sense signal.

### CROSS REFERENCE TO RELATED APPLICATIONS

This application claims priority of Taiwan Patent Application No. 112118258 filed on May 17, 2023, the entirety of which is incorporated by reference herein.

### BACKGROUND OF THE INVENTION

### Field of the Invention

The invention relates to a detection device, and more particularly, to a detection device and a detection method.

### Description of the Related Art

In recent years, technology to detect physiological information (Vital Signs) has seen rapid development (e.g., body temperature measurement, blood oxygen concentration detection, and non-contact radar detection devices for heartbeat and respiratory rates). These have been gradually applied in patient care, long-term care for the elderly, and infant care. In addition to the aforementioned physiological information, the measurement of body temperature is also an important issue for patients, the elderly, and infants who need to keep a record of body temperature. Today, nursing manpower is not sufficient, and there is an urgent need to provide detection equipment for assisting in this care.

### BRIEF SUMMARY OF THE INVENTION

In an exemplary embodiment, the invention is directed to a detection device for detecting an object with a sensor. The detection device includes a transmitter module, a receiver module, a processing module, and a computing module. The transmitter module transmits an RF (Radio Frequency) signal to the object. The receiver module receives a reflective signal from the object. The reflective signal includes a feature signal and a sense signal. The processing module converts the reflective signal into an integrated digital signal. The computing module determines the integrated digital signal into first digital information and second digital information. The first digital information corresponds to the feature signal. The second digital information corresponds to the sense signal.

In some embodiments, the sensor is an SAW (Surface Acoustic Wave) sensor.

In some embodiments, the computing module performs a first signal processing procedure on the integrated digital signal so as to determine the first digital information. The computing module further performs a second signal processing procedure on the integrated digital signal so as to determine the second digital information.

In some embodiments, after the receiver module receives the feature signal, the receiver module receives the sense signal.

In some embodiments, the processing module includes a demodulator and a sampler. The demodulator converts the reflective signal into a demodulation signal. The sampler converts the demodulation signal into the integrated digital signal.

In some embodiments, the computing module determines the integrated digital signal into the first digital information and the second digital information according to a partition time point.

In some embodiments, the first digital information is before the partition time point, and the second digital information is after the partition time point.

In some embodiments, the partition time point is about 300 ns to 500 ns after the transmitter module transmits the RF signal.

In some embodiments, the first digital information includes information related to breath and/or heartbeat.

In some embodiments, the second digital information includes information related to temperature, humidity, pressure, and/or chemical composition.

In an exemplary embodiment, the invention is directed to a detection method that includes the steps of: transmitting an RF signal to an object, wherein the object has a sensor; receiving a reflective signal from the object, wherein the reflective signal includes a feature signal and a sense signal; converting the reflective signal into an integrated digital signal; and determining the integrated digital signal into first digital information and second digital information, wherein the first digital information corresponds to the feature signal, and the second digital information corresponds to the sense signal.

In some embodiments, the detection method further includes: performing a first signal processing procedure on the integrated digital signal so as to determine the first digital information; and performing a second signal processing procedure on the integrated digital signal so as to determine the second digital information.

In some embodiments, the detection method further includes: after the feature signal is received, receiving the sense signal.

In some embodiments, the detection method further includes: converting the reflective signal into a demodulation signal; and converting the demodulation signal into the integrated digital signal.

In some embodiments, the detection method further includes: determining the integrated digital signal into the first digital information and the second digital information according to a partition time point.

In some embodiments, the partition time point is about 300 ns to 500 ns after the RF signal is transmitted.

### BRIEF DESCRIPTION OF DRAWINGS

The invention can be more fully understood by reading the subsequent detailed description and examples with references made to the accompanying drawings, wherein:

FIG. 1 is a diagram of a detection device according to an embodiment of the invention;

FIG. 2 is a diagram of a sensor according to an embodiment of the invention;

FIG. 3 is a diagram of a detection device according to an embodiment of the invention;

FIG. 4 is a diagram of a waveform of an integrated digital signal according to an embodiment of the invention;

FIG. 5 is a diagram of a time axis according to an embodiment of the invention;

FIG. 6 is a diagram of a time axis according to an embodiment of the invention; and

FIG. 7 is a flowchart of a detection method according to an embodiment of the invention.

### DETAILED DESCRIPTION OF THE INVENTION

In order to illustrate the foregoing and other purposes, features and advantages of the invention, the embodiments and figures of the invention will be described in detail as follows.

Certain terms are used throughout the description and following claims to refer to particular components. As one skilled in the art will appreciate, manufacturers may refer to a component by different names. This document does not intend to distinguish between components that differ in name but not function. In the following description and in the claims, the terms “include” and “comprise” are used in an open-ended fashion, and thus should be interpreted to mean “include, but not limited to . . . ”. The term “substantially” means the value is within an acceptable error range. One skilled in the art can solve the technical problem within a predetermined error range and achieve the proposed technical performance. Also, the term “couple” is intended to mean either an indirect or direct electrical connection. Accordingly, if one device is coupled to another device, that connection may be through a direct electrical connection, or through an indirect electrical connection via other devices and connections.

The following disclosure provides many different embodiments, or examples, for implementing different features of the subject matter provided. Specific examples of components and arrangements are described below to simplify the present disclosure. These are, of course, merely examples and are not intended to be limiting. For example, the formation of a first feature over or on a second feature in the description that follows may include embodiments in which the first and second features are formed in direct contact, and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed.

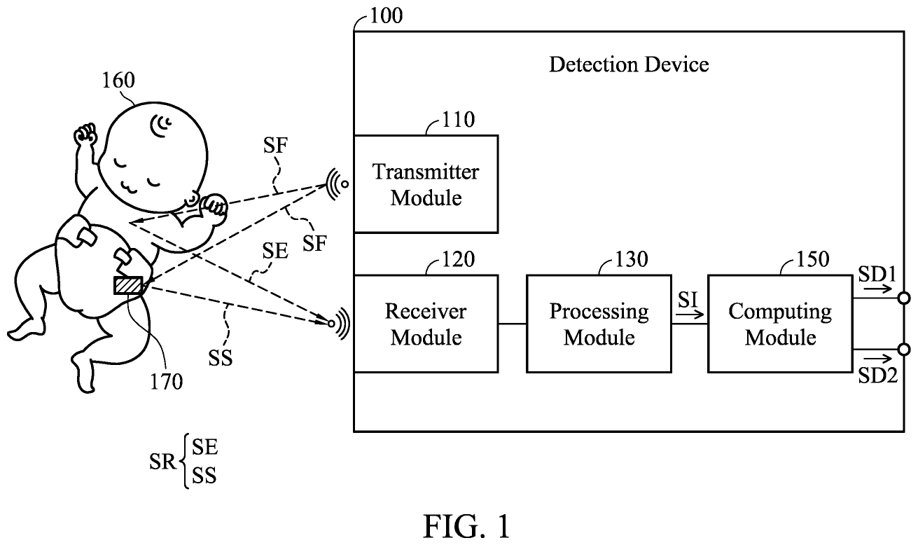

FIG. 1 is a diagram of a detection device 100 according to an embodiment of the invention. The detection device 100 may be applied to a radar device, such as an IR-UWB (Impulse Radio Ultra-Wideband) radar, but it is not limited thereto. In the embodiment of FIG. 1, the detection device 100 includes a transmitter module 110, a receiver module 120, a processing module 130, and a computing module 150. It should be understood that the detection device 100 may further include other components, such as a housing, a controller, and/or a power supply module, although they are not displayed in FIG. 1.

In some embodiments, the detection device 100 is configured to detect an object 160 with a sensor 170. It should be noted that both the object 160 and the sensor 170 are external elements, and they are not any portions of the detection device 100. For example, the aforementioned object 160 may be a human body, and the sensor 170 may be disposed on/at any position of the aforementioned human body, but they are not limited thereto.

The transmitter module 110 can transmit an RF (Radio Frequency) signal SF to the object 160. In some embodiments, the operational frequency of the RF signal SF is from 3.1 GHz to 10.6 GHz, so as to support the UWB operations. In response, the receiver module 120 can receive a reflective signal SR from the object 160. The reflective signal SR includes a feature signal SE and a sense signal SS. Specifically, the feature signal SE may include the information related to the object 160, and the sense signal SS may include the information related to the sensor 170. For example, when the object 160 receives the RF signal SF, the object 160 can transmit the corresponding feature signal SE back. In some embodiments, the feature signal SE is a physiological signal.

The processing module 130 is coupled to the receiver module 120. The processing module 130 can convert the reflective signal SR into an integrated digital signal SI. The computing module 150 is coupled to the processing module 130. The computing module 150 can determine the integrated digital signal SI into first digital information SD1 and second digital information SD2. It should be noted that the first digital information SD1 corresponds to the feature signal SE, and the second digital information SD2 corresponds to the sense signal SS.

Specifically, the computing module 150 can perform a first signal processing procedure on the integrated digital signal SI, so as to determine the first digital information SD1. In some embodiments, the first signal processing procedure includes a variety of algorithms, such as time-domain processing, frequency-domain processing, or AI (Artificial Intelligence) machine learning. The first digital information SD1, extracted from the integrated digital signal SI, may include the information related to breath and/or heartbeat, but it is not limited thereto.

In some embodiments, the aforementioned AI algorithms include supervised learning, un-supervised learning, semi-supervised learning, or reinforcement learning.

Furthermore, the computing module 150 can perform a second signal processing procedure on the integrated digital signal SI, so as to determine the second digital information SD2. In some embodiments, the second signal processing procedure includes a variety of algorithms, such as time-domain processing or AI machine learning. The second digital information SD2, extracted from the integrated digital signal SI, may include the information related to temperature, humidity, pressure, and/or chemical composition, but it is not limited thereto.

Generally, the detection device 100 merely uses the same transmitter module 110 and the same receiver module 120 to obtain two different types of information, and it effectively simplifies the overall circuit complexity and reduces the relative manufacturing cost. With such a design of the invention, the computing module 150 of the detection device 100 can also separate the two different types of information, so as to improve the following analysis and process.

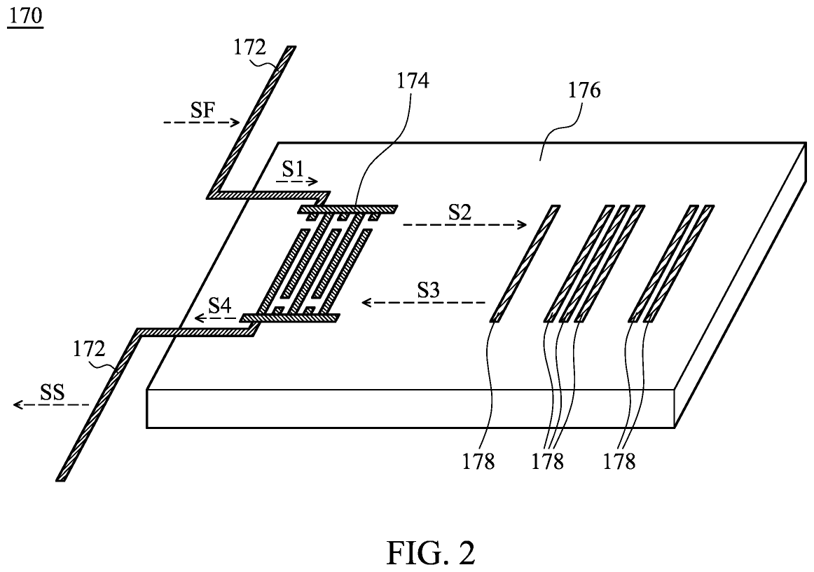

FIG. 2 is a diagram of the sensor 170 according to an embodiment of the invention. In the embodiment of FIG. 2, the sensor 170 is a passive SAW (Surface Acoustic Wave) sensor which includes an antenna module 172, an IDT (Interdigital Transducer) 174, a piezoelectric substrate 176, and one or more reflectors 178. For example, when the sensor 170 receives the RF signal SF, it can transmit the corresponding sense signal SS back.

In the beginning, the antenna module 172 can receive the RF signal SF, and generate a first electric signal S1 according to the RF signal SF. The IDT 174 is coupled to the antenna module 172, and is disposed on the piezoelectric substrate 176. Because of the reverse piezoelectric effect of the piezoelectric substrate 176, the IDT 174 can convert the first electric signal S1 into a first SAW signal S2. Next, the reflectors 178 can reflect the first SAW signal S2, so as to generate a second SAW signal S3. The second SAW signal S3 and the first SAW signal S2 may have exactly opposite directions of propagation. Because of the piezoelectric effect of the piezoelectric substrate 176, the IDT 174 can convert the second SAW signal S3 into a second electric signal S4. Finally, the antenna module 172 can receive the second electric signal S4, and generate and transmit the aforementioned sense signal SS to the detection device 100 according to the second electric signal S4.

It should be noted that the speed of propagation of the RF signal SF and the reflective signal SR is almost equal to the speed of light. However, the speed of propagation of the first SAW signal S2 and the second SAW signal S3 is only substantially equal to the speed of sound. The speed of sound is also affected by a surrounding temperature. Thus, there may be a relatively long delay time between the sense signal SS and the RF signal SF. In addition, because the feature signal SE has nothing to do with any SAW signals, the reception time point of the feature signal SE is usually earlier than the reception time point of the sense signal SS. In some embodiments, the computing module 150 of the detection device 100 can estimate a possible temperature of the sensor 170 by analyzing the delay time of the sense signal SS. The possible temperature of the sensor 170 may be substantially the same as the current temperature of the object 160. In some embodiments, if the sensor 170 includes more reflectors 178, they can provide a plurality of delay times, so as to improve the accuracy of the aforementioned temperature estimation.

The following embodiments will introduce different configurations and detail structural features of the detection device 100. It should be understood that these figures and descriptions are merely exemplary, rather than limitations of the invention.

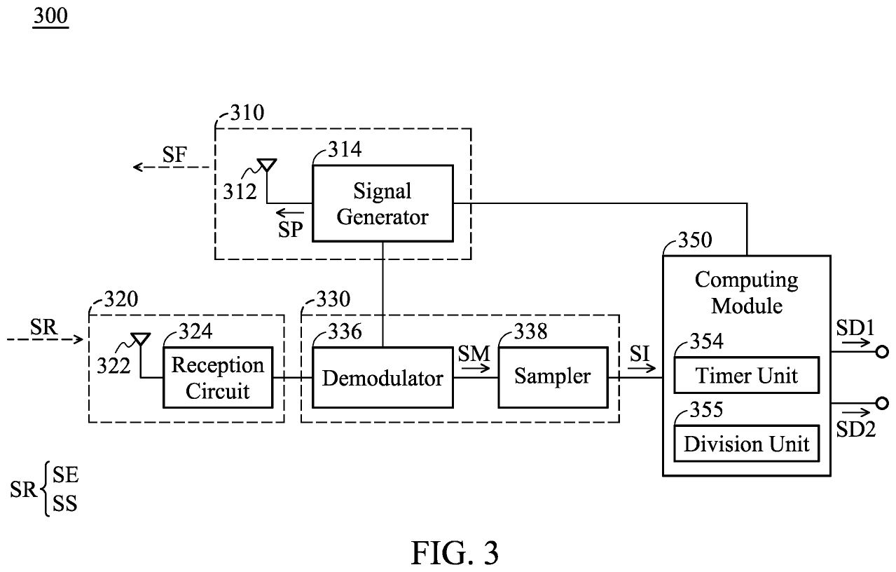

FIG. 3 is a diagram of a detection device 300 according to an embodiment of the invention. FIG. 3 is similar to FIG. 1. In the embodiment of FIG. 3, the detection device 300 includes a transmitter module 310, a receiver module 320, a processing module 330, and a computing module 350.

The transmitter module 310 includes a transmission antenna 312 and a signal generator 314. The transmission antenna 312 is coupled to the signal generator 314. The signal generator 314 can generate an impulse signal SP. The transmission antenna 312 can generate and transmit an RF signal SF according to the impulse signal SP. The receiver module 320 includes a reception antenna 322 and a reception circuit 324. The reception circuit 324 is coupled to the reception antenna 322. The reception antenna 322 can receive a reflective signal SR. The reflective signal SR may include a feature signal SE and a sense signal SS. For example, the reception circuit 324 may include an LNA (Low Noise Amplifier) and/or an LPF (Low-Pass Filter), but it is not limited thereto.

As mentioned above, there is usually a relatively long delay time between the sense signal SS and the RF signal SF. Thus, in some embodiments, after the reception antenna 322 of the receiver module 320 receives the feature signal SE, the reception antenna 322 of the receiver module 320 can receive the sense signal SS.

The shapes and types of the transmission antenna 312 and the reception antenna 322 are not limited in the invention. In some embodiments, any of the transmission antenna 312 and the reception antenna 322 is a monopole antenna, a dipole antenna, a patch antenna, a loop antenna, a PIFA (Planar Inverted F Antenna), or a chip antenna.

The processing module 330 includes a demodulator 336 and a sampler 338. The demodulator 336 is coupled to the signal generator 314 and the reception circuit 324. The demodulator 336 can convert the reflective signal SR into a demodulation signal SM. The sampler 338 is coupled to the demodulator 336. The sampler 338 can convert the demodulation signal SM into an integrated digital signal SI. It should be noted that the sampling rate of the sampler 338 is very high, and the operational speed thereof is significantly higher than that of a general ADC (Analog-to-Digital Converter). In some embodiments, the sampler 338 uses the technology of CTBV (Continuous Time Binary Value) to convert the demodulation signal SM into the integrated digital signal SI, but it is not limited thereto. In some embodiments, the demodulator 336 is integrated with the sampler 338, so as to form a single circuit.

The computing module 350 is coupled to the processing module 330. The integrated digital signal SI, which is received by the computing module 350, includes first digital information SD1 and second digital information SD2. In some embodiments, the computing module 350 includes a timer unit 354 and a division unit 355, whose functions will be described in detail over the following embodiments. It should be understood that the timer unit 354 and the division unit 355 may be implemented with hardware circuits or software programs.

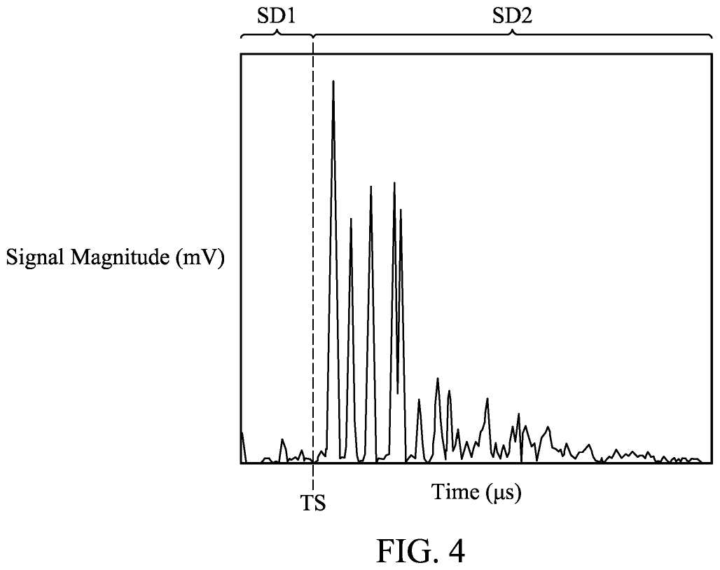

FIG. 4 is a diagram of a waveform of the integrated digital signal SI according to an embodiment of the invention. The horizontal axis represents time (s), and the vertical axis represents signal magnitude (mV). As mentioned above, the first digital information SD1 corresponds to the feature signal SE, and the second digital information SD2 corresponds to the sense signal SS. Since the reception time point of the sense signal SS is usually later than the reception time point of the feature signal SE, the computing module 350 can determine the integrated digital signal SI into the first digital information SD1 and the second digital information SD2 according to a partition time point TS.

Specifically, the first digital information SD1 may be before the partition time point TS, and the second digital information SD2 may be after the partition time point TS. Initially, the timer unit 354 of the computing module 350 can calculate the accurate partition time point TS. Next, the division unit 355 of the computing module 350 can use the partition time point TS to determine the integrated digital signal SI into front and back portions, where the front portion is considered as the first digital information SD1, and the back portion is considered as the second digital information SD2. In some embodiments, the partition time point TS is about 300 ns to 500 ns after the transmitter module 310 transmits the RF signal SF. For example, if the transmitter module 310 transmits the RF signal SF exactly at 0 ns, the partition time point TS may be set within a range from 300 ns to 500 ns. The possible range of the partition time point TS is calculated and obtained according to many experimental results, and it helps to improve the accuracy of signal determination of the computing module 350 of the detection device 300.

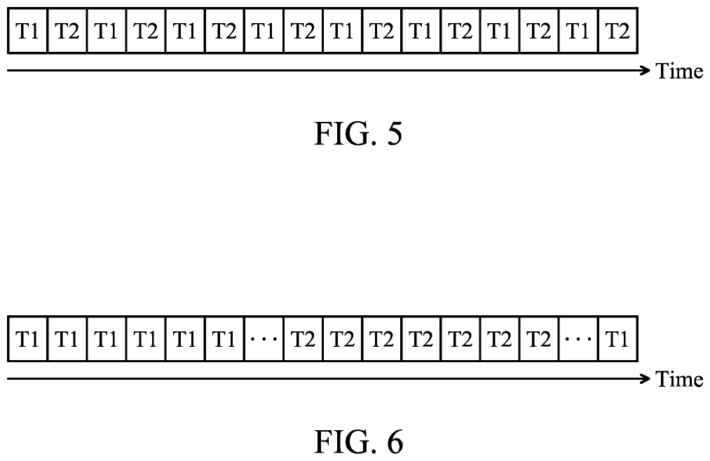

FIG. 5 is a diagram of a time axis according to an embodiment of the invention. As shown in FIG. 5, the time axis includes a plurality of first intervals T1 and a plurality of second intervals T2. The second intervals T2 are interleaved with the first intervals T1. In the embodiment of FIG. 5, the detection device 100 can transmit the RF signal SF within each first interval T1, and then receive the feature signal SE from the object 160. As mentioned above, after the received signal is sampled, the first digital information SD1 can be determined by performing the first signal processing procedure, and the first digital information SD1 can correspond to the feature signal SE. In some embodiments, the first signal processing procedure includes a variety of algorithms, such as time-domain processing, frequency-domain processing, or AI machine learning. The first digital information SD1, extracted from the integrated digital signal SI, may include the information related to breath and/or heartbeat, but it is not limited thereto.

In addition, the detection device 100 can also transmit the RF signal SF within each second interval T2, and then receive the sense signal SS from the sensor 170. As mentioned above, after the received signal is sampled, the second digital information SD2 can be determined by performing the second signal processing procedure, and the second digital information SD2 can correspond to the sense signal SS. In some embodiments, the second signal processing procedure includes a variety of algorithms, such as time-domain processing or AI machine learning. The second digital information SD2, extracted from the integrated digital signal SI, may include the information related to temperature, humidity, pressure, and/or chemical composition, but it is not limited thereto.

In conclusion, the detection device 100 can use a design of time multiplexing to alternately receive, sample and compute the feature signal SE and the sense signal SS as mentioned above.

FIG. 6 is a diagram of a time axis according to an embodiment of the invention. FIG. 6 is similar to FIG. 5. As shown in FIG. 6, the time axis includes a plurality of first intervals T1 and a plurality of second intervals T2. The first intervals T1 can appear continuously, and the second intervals T2 can also appear continuously, but they are not limited thereto. Similarly, the detection device 100 can also use another design of time multiplexing of FIG. 6 to alternately receive, sample and compute the feature signal SE and the sense signal SS as mentioned above.

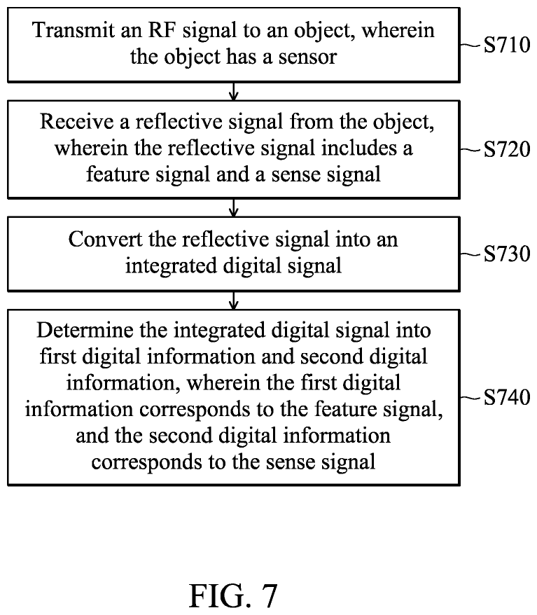

FIG. 7 is a flowchart of a detection method according to an embodiment of the invention. To begin, in step S710, an RF signal is transmitted to an object. The object has a sensor. In step S720, a reflective signal is received from the object. The reflective signal includes a feature signal and a sense signal. In step S730, the reflective signal is converted into an integrated digital signal. Finally, in step S740, the integrated digital signal is determined into first digital information and second digital information. The first digital information corresponds to the feature signal. The second digital information corresponds to the sense signal. It should be understood that these steps are not required to be performed in order, and every feature of the embodiments of FIGS. 1 to 6 may be applied to the detection method of FIG. 7.

The invention proposes a novel detection device and a novel detection method. In comparison to the conventional design, the invention has at least the advantages of simplifying the overall circuit complexity and reducing the relative manufacturing cost. Therefore, the invention is suitable for application in a variety of devices.

Note that the above element parameters are not limitations of the invention. A designer can fine-tune these setting values according to different requirements. It should be understood that the detection device and detection method of the invention are not limited to the configurations of FIGS. 1-7. The invention may include any one or more features of any one or more embodiments of FIGS. 1-7. In other words, not all of the features displayed in the figures should be implemented in the detection device and detection method of the invention.

The method of the invention, or certain aspects or portions thereof, may take the form of program code (i.e., executable instructions) embodied in tangible media, such as floppy diskettes, CD-ROMS, hard drives, or any other machine-readable storage medium, wherein, when the program code is loaded into and executed by a machine such as a computer, the machine thereby becomes an apparatus for practicing the methods. The methods may also be embodied in the form of program code transmitted over some transmission medium, such as electrical wiring or cabling, through fiber optics, or via any other form of transmission, wherein, when the program code is received and loaded into and executed by a machine such as a computer, the machine becomes an apparatus for practicing the disclosed methods. When implemented on a general-purpose processor, the program code combines with the processor to provide a unique apparatus that operates analogously to application-specific logic circuits.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having the same name (but for use of the ordinal term) to distinguish the claim elements.

It will be apparent to those skilled in the art that various modifications and variations can be made in the invention. It is intended that the standard and examples be considered as exemplary only, with a true scope of the disclosed embodiments being indicated by the following claims and their equivalents.

What is claimed is:

1. A detection device for detecting an object with a sensor, comprising:
a transmitter module, transmitting an RF (Radio Frequency) signal to the object;
a receiver module, receiving a reflective signal from the object, wherein the reflective signal comprises a feature signal and a sense signal;
a processing module, converting the reflective signal into an integrated digital signal; and
a computing module, determining the integrated digital signal into first digital information and second digital information;
wherein the first digital information corresponds to the feature signal, and the second digital information corresponds to the sense signal.

2. The detection device as claimed in claim 1, wherein the sensor is an SAW (Surface Acoustic Wave) sensor.

3. The detection device as claimed in claim 1, wherein the computing module performs a first signal processing procedure on the integrated digital signal so as to determine the first digital information, and the computing module further performs a second signal processing procedure on the integrated digital signal so as to determine the second digital information.

4. The detection device as claimed in claim 1, wherein after the receiver module receives the feature signal, the receiver module receives the sense signal.

5. The detection device as claimed in claim 1, wherein the processing module comprises:
a demodulator, converting the reflective signal into a demodulation signal; and
a sampler, converting the demodulation signal into the integrated digital signal.

6. The detection device as claimed in claim 1, wherein the computing module determines the integrated digital signal into the first digital information and the second digital information according to a partition time point.

7. The detection device as claimed in claim 6, wherein the first digital information is before the partition time point, and the second digital information is after the partition time point.

8. The detection device as claimed in claim 6, wherein the partition time point is about 300 ns to 500 ns after the transmitter module transmits the RF signal.

9. The detection device as claimed in claim 1, wherein the first digital information comprises information related to breath and/or heartbeat.

10. The detection device as claimed in claim 1, wherein the second digital information comprises information related to temperature, humidity, pressure, and/or chemical composition.

11. A detection method, comprising the steps of:
transmitting an RF signal to an object, wherein the object has a sensor;
receiving a reflective signal from the object, wherein the reflective signal comprises a feature signal and a sense signal;
converting the reflective signal into an integrated digital signal; and
determining the integrated digital signal into first digital information and second digital information, wherein the first digital information corresponds to the feature signal, and the second digital information corresponds to the sense signal.

12. The detection method as claimed in claim 11, wherein the sensor is an SAW sensor.

13. The detection method as claimed in claim 11, further comprising:
performing a first signal processing procedure on the integrated digital signal so as to determine the first digital information; and
performing a second signal processing procedure on the integrated digital signal so as to determine the second digital information.

14. The detection method as claimed in claim 11, further comprising:
after the feature signal is received, receiving the sense signal.

15. The detection method as claimed in claim 11, further comprising:
converting the reflective signal into a demodulation signal; and
converting the demodulation signal into the integrated digital signal.

16. The detection method as claimed in claim 11, further comprising:
determining the integrated digital signal into the first digital information and the second digital information according to a partition time point.

17. The detection method as claimed in claim 16, wherein the first digital information is before the partition time point, and the second digital information is after the partition time point.

18. The detection method as claimed in claim 16, wherein the partition time point is about 300 ns to 500 ns after the RF signal is transmitted.

19. The detection method as claimed in claim 11, wherein the first digital information comprises information related to breath and/or heartbeat.

20. The detection method as claimed in claim 11, wherein the second digital information comprises information related to temperature, humidity, pressure, and/or chemical composition.
